(19) United States
(12) Patent Application Publication
(10) Pub. No.: US 2022/0111157 A1
(43) Pub. Date: Apr. 14, 2022

(54) ENHANCED SYSTEM AND METHOD FOR USING A PRESSURIZED HYALURONIC ACID INJECTION DEVICE FOR AESTHETIC ENHANCEMENT OF SKIN APPEARANCE

(71) Applicant: MAYRA JIMENEZ, Riverside, CA (US)

(72) Inventor: MAYRA JIMENEZ, Riverside, CA (US)

(21) Appl. No.: 17/472,426

(22) Filed: Sep. 10, 2021

Related U.S. Application Data
(60) Provisional application No. 63/077,030, filed on Sep. 11, 2020.

(51) Int. Cl. A61M 5/46; A61K 8/73; A61Q 19/08; A61L 27/20

(52) U.S. Cl. CPC A61M 5/46; A61K 8/735; A61Q 19/08; A61L 27/20; A61M 2205/52; A61K 2800/87; A61L 2430/34; A61M 2210/04; A61M 2210/0606; A61K 2800/91

(57) ABSTRACT

The present invention is a handheld device used to deliver hyaluronic acid to the skin and is used in the dermatology field and as an aesthetic treatment. The present invention uses pressure to launch a hyaluronic acid mixture into the skin that is better tolerated and less painful than conventional hypodermic needle procedures. The present invention is a non-invasive tool that delivers hyaluronic acid into the skin through the use of pressure as opposed to a hypodermic needle, thereby significantly decreasing the likelihood of damage to the skin when compared to a hypodermic needle device.

### PRIORITY CLAIMS

This application claims the benefit of U.S. Provisional Patent Application Ser. No. 63/077,030, filed on Sep. 11, 2020, the contents of which are incorporated herein.

### FIELD OF THE INVENTION

The present invention is related to a system and method for using a handheld pressurized hyaluronic acid injection device, or “hyaluron pen”, to treat a wide variety of dermatological concerns.

### BACKGROUND OF THE INVENTION

Hyaluronic acid (“HA”) is a gel-like water-holding molecule with a moisture-binding ingredient that helps keep skin plump and hydrated. Approximately 50% of the body's total HA is in the skin. HA acts as a scaffold for the extracellular matrix, providing rigidity, hydration and pressure while allowing cellular movement and regeneration. It is also important in protecting the skin from free radical damage, particularly against UVA and UVB. HA is rapidly metabolized in the tissues, with one third of the HA in the human body being metabolized daily. With its ability to fill and plump up the extracellular tissue space in the skin, HA also adds a range of skin-boosting benefits. It has become an increasingly popular ingredient in serums and moisturizers for its superpower hydrating and anti-aging properties; the HA molecule can hold up to 1000 times its weight in water. In time, the body produces fewer HA molecules to bind with water and the collagen is stretched and becomes less hydrated which results in lines and wrinkles. The more HA there is inside the epidermis and dermis layers of the skin, the more water is retained. This results in better moisturized and youthful-looking skin.

The most popular HA fillers on the market are Juvederm and Restylane. Juvederm is a HA-based dermal filler. There are several products in the Juvederm family, all of which help address the facial signs of aging. Each product in the Juvederm line features different bonding and HA concentrations. Juvederm is formulated with lidocaine and saline. The different products are each tailored to target specific problems when injected into different areas and depths. Juvederm fillers are made of non-animal stabilized hyaluronic acid created in laboratories from bacterial cultures. Restylane is an injected gel that contains HA, helping to plump up the skin and was designed to work like the natural HA in the body, improving the appearance of fine lines and wrinkles. Restylane is formulated with lidocaine and saline.

Needle-free devices have been utilized extensively in healthcare for decades. The first air-powered needle-free systems, originally called jet injectors, were developed in the 1930s and used in vaccination for smallpox, polio and measles. In the field of dermatology, modern hyaluronic acid injection devices have been designed to deliver precise injections without the discomfort and anxiety of a hypodermic needle. This interest in needle-free technology has been steadily growing over the past few years.

It is understood that 20% of the population has some degree of fear of needles and 10% within that number suffer from Trypanophobia (“Trip-ano-phobia”), an excessive or irrational fear of injections or needles which can be learned or inherited.

### SUMMARY OF THE INVENTION

The present invention is a handheld device used to deliver hyaluronic acid to the skin and an overall method and method for using it. Beyond its use in healthcare, this technology has now been developed as an aesthetics treatment. The present invention uses pressure to launch a hyaluronic acid mixture into the skin that is better tolerated and less painful than conventional hypodermic needle procedures. The present invention is a non-invasive tool that delivers hyaluronic acid into the skin through the use of pressure as opposed to a hypodermic needle. The present invention significantly decreases the likelihood of damage to the skin when compared to a hypodermic needle device. In addition, many clients have an aversion to needles in general and the present invention satisfies clients' desires to receive cosmetic skin treatments without the use of needles.

The Hyaluronic acid fillers used in the hyaluron pen are comprised of a regular and dense monophasic structure, which makes the gel product stable and consistent. The consistency of the pattern allows for naturally harmonized volume, smooth insertion and promotes a stronger support system within the skin structure. The hyaluron pen has a low percentage of product migration, meaning that once inside the outermost layer of skin the product is more likely to stay close to the insertion site with accuracy. The hyaluron pen utilizes a unique cross-linking technology that results in increased and stabilized product longevity. The Hyaluronic acid Filler used in the hyaluron pen is a thick and longer lasting gel, used to treat deep wrinkles and nasolabial folds or augmentation of the cheeks, chin, and lips. The HA concentration is 24 mg/ml at a volume of 1.1 ml. The resulting duration of the treatment lasts 6-9 months. The hyaluron pen contour device has the thickest properties within the product line. With an advanced ability to mold, maintain structure and longevity, it is recommended for treatment of deep sized to extremely severe wrinkles including nasolabial and face, cheek and chin contours. The HA concentration is 24 mg/ml at a volume of 1.1 ml. The resulting duration of the treatment lasts up to 12 Months. According to the present invention, the concentration and viscosity of the gel may be varied depending upon the various skin conditions of clients and depending upon the aesthetic result desired by each client. The thickest properties may be reserved for the most severe skin anomalies including wrinkles, while the hyaluron acid filler may be varied in terms of the amount inserted under the outmost layer of skin, varied by client, and varied by region of skin and features of skin per client, and that amount and density may be varied by changing the rate at which the pen ejects the acid and in terms of the application pattern and rate employed by the clinician who is applying the hyaluron acid to the client by way of the pen.

The present invention implements a novel application method. The hyaluron pen device must be pressed firmly against skin. The surface of the skin must be kept flat during the treatment process, making sure its vacuum sealed against the outer layer of the skin. By slowing down this treatment process, more acid may be infused into the client's skin per square unit of measurement, and by pressing down more firmly, more acid similarly may be infused into the client's skin per square unit of measurement. Conversely, a faster, less forceful application will result in less of the acid infused per square unit of measurement of skin of the client.

The present invention can be used to treat dermatological concerns such as: firming and filling wrinkles, nasolabial folds, and marionette lines; and lip enhancement. This treatment typically lasts approximately from six to nine months and sometimes longer, depending on the hyaluronic acid used in the treatment and how quickly it breaks down in the recipient's skin. Finally, a camera may be used to observe clients before and after the application, as well as many days after the application, so clients can compare before, immediately after and relatively longer after application results, so that results may be measured and stored digitally. Subsequently, these stored results can be cumulatively saved, so that in training future clinical technicians, they can correlate prior results with application techniques including density of acid applied per square unit of client skin treated. In the alternative, a form of artificial intelligence may be applied during subsequent applications by client, by skin type, by skin anomalies and so forth, directing the clinical technician during the treatment process so that that clinical technician is directed via the artificially intelligent “guide”, even by oral directives, so that clinical technicians perform computer optimized procedures.

These and other aspects, objects, features and advantages of the present invention, are specifically set forth in, or will become apparent from, the following detailed description of an exemplary embodiment of the invention.

### BRIEF DESCRIPTION OF THE DRAWINGS

FIG. 1 is a diagram of the hyaluron pen device of the present invention.

FIG. 2 is an image of the hyaluron pen system of the present invention.

FIG. 3 is a diagram of the hyaluronic acid dermal application method of present invention compared to a conventional hypodermic needle-based application method, along with a computer system for use with and incorporation into the present invention.

FIG. 4 is a diagram of the primary facial application areas of the present invention.

FIG. 5 is a diagram of facial anatomy and physiology pertaining to bone structure as used in the training process of the present invention.

FIG. 6 is a diagram of facial anatomy and physiology pertaining to the regions of the face as used in the training process of the present invention.

FIGS. 7A-B are diagrams of the internal and external structure of skin as used in the training process of the present invention.

FIGS. 8A-I are images of common skin disorders as used in the training process of the present invention.

FIG. 9 is a hand washing diagram as used in the training process of the present invention.

### DETAILED DESCRIPTION OF THE PREFERRED EMBODIMENT

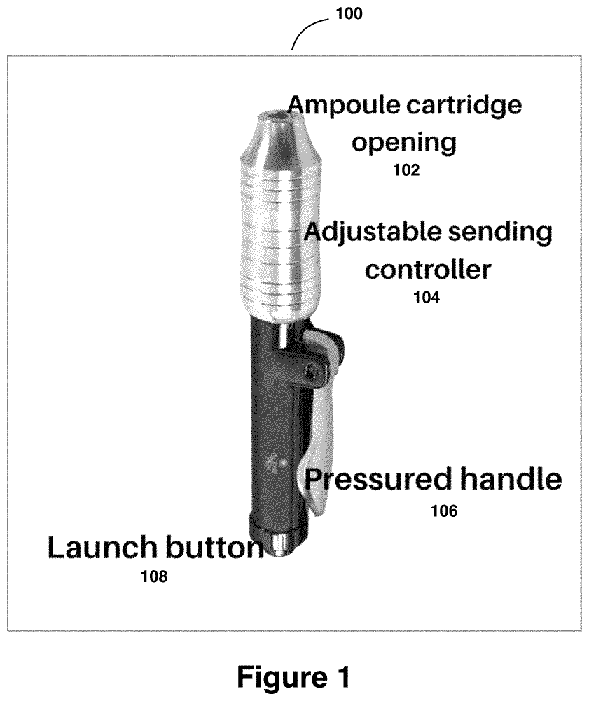

FIG. 1 is a diagram of the hyaluron pen device of the present invention. In accordance with the preferred embodiment of the present invention, the hyaluron pen 100, as shown in FIG. 1, is a handheld device that is comprised of: an ampoule cartridge opening; an adjustable sending controller; a pressurized handle to manually control the necessary pressure levels for optimal application; and a launch button to initiate the application of the Hyaluronic acid to the skin. The hyaluron pen 100 is a non-invasive tool that delivers Hyaluronic acid into the skin by pressure and not via a hypodermic needle. It is understood that 20% of the population has some degree of fear of needles and 10% within that number suffer from Trypanophobia (“Trip-ano-phobia”), an excessive or irrational fear of injections or needles which can be learned or inherited. The hyaluron pen 100 is designed to deliver precise injections without the discomfort and anxiety of a hypodermic needle. The hyaluron pen 100 produces just enough pressure to launch a Hyaluronic acid mixture into the skin, in much more comfortable and stress-free way than conventional hypodermic needle procedures. Using the hyaluron pen 100, any fear or stigma of undergoing a needle-based procedure is instantly removed and when undergoing a hyaluron pen 100 treatment for the first time, the treatment recipient may notice that the pain is surprisingly (and impressively) minimal. Coupled with the proper training techniques for using the hyaluron pen 100, the likelihood of causing severe damage such as skin necrosis as a result of a hyaluron pen 100 is treatment is greatly reduced in comparison to a hypodermic needle treatment.

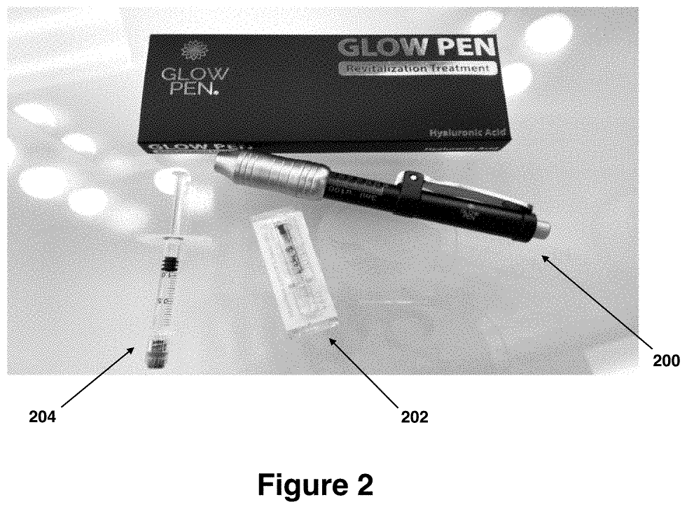

FIG. 2 is an image of the hyaluron pen system of the present invention. In accordance with the preferred embodiment of the present invention, the system uses a combination of parts as shown in FIG. 2. The key parts to the system of the present invention are: a handheld hyaluron pen device 200; a disposable single use Hyaluronic acid ampoule cartridge 202; and a syringe applicator to insert the ampoule cartridge into the handheld hyaluron pen device 204. The hyaluron pen device uses 1 ampoule cartridge 202 per treatment, which is disposed after the treatment session. The syringe applicator 204 is a three-tenths milliliter (Cartridge Size) syringe. It may be called a “0.3 mL” syringe or “0.3 cc” syringe. It is also known as an insulin syringe. Although it is labeled in “units” at the bottom of the syringe, each unit actually is one-hundredth of a milliliter (0.01 mL or 0.01 cc). Each small black mark on the syringe equals 0.01 ml. A larger black mark and a number is found every 0.05 mL (i.e. five-hundredths of a ml). A measurement of 0.5 mL results in 12 shots of Hyaluronic acid, and a measurement of 1.0 mL results in 24 shots. The Hyaluronic acid is inserted slowly into the cartridge 202 until it reaches 15 (as marked) on the cartridge 202. The syringe 204 plunger is then placed back into the cartridge 202 (to the 30 black mark on cartridge) so there is no loss of Hyaluronic acid. The 0.3 mL cartridge 202 capacity results in 6 shots of Hyaluronic acid, after which the cartridge 202 is re-loaded if more shots are necessary to complete the treatment.

When choosing hyaluronic acid (HA), it is best to select a fine, non animal-based filler with a high HA mg ratio. It is also important to choose a non-toxic, non-permanent

HA and to ensure that the HA is obtained from a reputable company. The basic properties of a good HA can be categorized into three elements. elasticity, which is the ability of the HA to return to its initial shape after some sort of strain is applied; cohesiveness, which is the ability of the HA to remain intact and cohesive, to keep its integrity; and plasticity, which is the ability of the HA to be modified and its malleableness. Raw HA mixture is unique as it contains the following properties: total HA concentration; amount of soluble HA; and average molecular weight (MW) and HA. Cross-linking decreases the percentage of free-floating HA and makes the product more gel-like. This is believed to prolong the treatment results more than the concentration of HA alone. The elasticity of the HA is dependent upon the size of the particles of HA and the technician will need to consider the following factors: the gel and fluid HA ratio; the gel hardness and viscosity; and the gel expansion (resulting in post-treatment swelling).

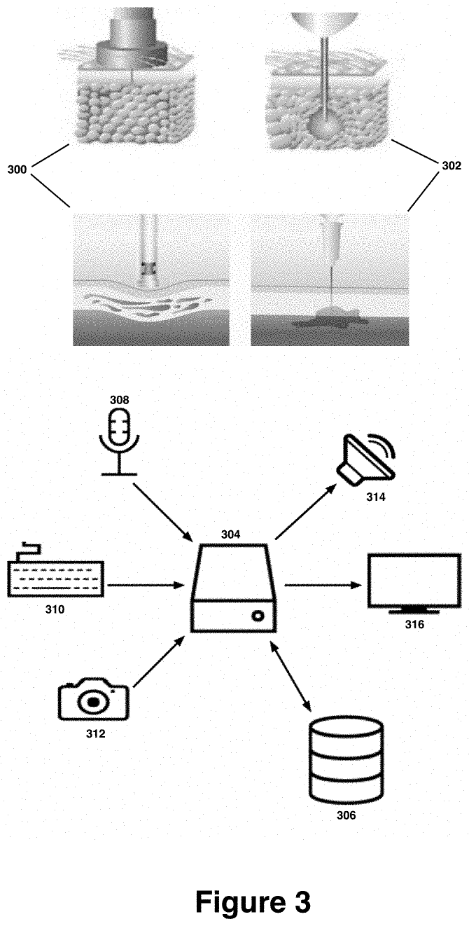

FIG. 3 is a diagram of the hyaluronic acid dermal application method of present invention compared to a conventional hypodermic needle-based application method, along with a computer system for use with and incorporation into the present invention. In accordance with the preferred embodiment, the present invention uses pressure to launch a hyaluronic acid mixture into the skin 300 that is better tolerated and less painful than conventional hypodermic needle procedures 302. The present invention delivers hyaluronic acid into the skin using a non-invasive pressure injection method 300 as opposed to a hypodermic needle 302, thereby significantly decreasing the likelihood of damage to the skin. The hyaluronic acid fillers used in the hyaluron pen are comprised of a regular and dense monophasic structure, which makes the gel product stable and consistent. The consistency of the pattern allows for naturally harmonized volume, smooth insertion and promotes a stronger support system within the skin structure. The hyaluron pen has a low percentage of product migration, meaning that once inside the product is more likely to stay close to the insertion site with accuracy. The hyaluron pen utilizes a unique cross-linking technology that results in increased and stabilized product longevity. The hyaluronic acid filler used in the hyaluron pen is a thick and longer lasting gel, used to treat deep wrinkles and nasolabial folds or augmentation of the cheeks, chin, and lips. The HA concentration is 24 mg/ml at a volume of 1.1 ml. The resulting duration of the treatment lasts 6-9 months. The hyaluron pen contour device has the thickest properties within the product line. With an advanced ability to mold, maintain structure and longevity, it is recommended for treatment of deep sized to extremely severe wrinkles including nasolabial and face, cheek and chin contours. The HA concentration is 24 mg/ml at a volume of 1.1 ml. The resulting duration of the treatment lasts up to 12 Months.

The present invention can be used to treat dermatological concerns such as: firming and filling wrinkles, nasolabial folds, and marionette lines; and lip enhancement. This treatment typically lasts approximately from six to nine months and sometimes longer, depending on the hyaluronic acid used in the treatment and how quickly it breaks down in the recipient's skin.

Finally, a camera 312 may be used to observe clients before and after the application, as well as many days after the application, so clients can compare before, immediately after and relatively longer after application results, so that results may be measured and stored digitally. Subsequently, these stored results can be cumulatively saved, so that in training future clinical technicians, they can correlate prior results with application techniques including density of acid applied per square unit of client skin treated. In the alternative, a form of artificial intelligence may be applied during subsequent applications by client, by skin type, by skin anomalies and so forth, directing the clinical technician during the treatment process so that that clinical technician is directed via the artificially intelligent “guide”, even by oral directives, so that clinical technicians perform computer optimized procedures.

As shown in FIG. 3, a computer 304 may be used to keep track of a clinical technician's clients. Multiple clinics and technicians may be interconnected by way of a database 306. A microphone 308 and keyboard 310 may be connected to the computer 304 so a clinician may annotate any procedure performed by that clinician, and as well, a camera 312 of suitable resolution may be employed by a clinician to record various skin conditions presented by a client, and a monitor or display 316 may be connected to the computer 304 to recall various skin conditions, or as a visual aid for a clinician so that a clinician may be directed with on-screen directions. Also, a speaker 314 may be employed connected to the computer 304 so that a clinician may be directed during a procedure. For example: assert more pressure on the pen in a certain region of a particular client; move the pen faster or slower in a certain region of a particular client, all the while comparing data from the database 306, both in terms of that actual client and other similar clients with similar skin conditions (e.g., color, tone, pigment, wrinkles and extent of wrinkles, moles, etc.). As more and more data is accumulated according to the present invention, clinicians and systems of clinicians may develop multiple databases 306, and the databases 306 may even be accessible by clients, showing them before and after and well-after images, as part of marketing services and demonstrating the efficacy of the present system and method according to the present invention. The result of this configuration would be like an “Alexa” for interacting with a clinical technician, being able to take down clinician notes for client archives and for being able to help instruct a clinician during a client procedure, based on norms, client norms, a wide range of client norms, and conditions presented to a clinician, which may be spoken to the computer 304 via microphone 308 or input via keyboard 310.

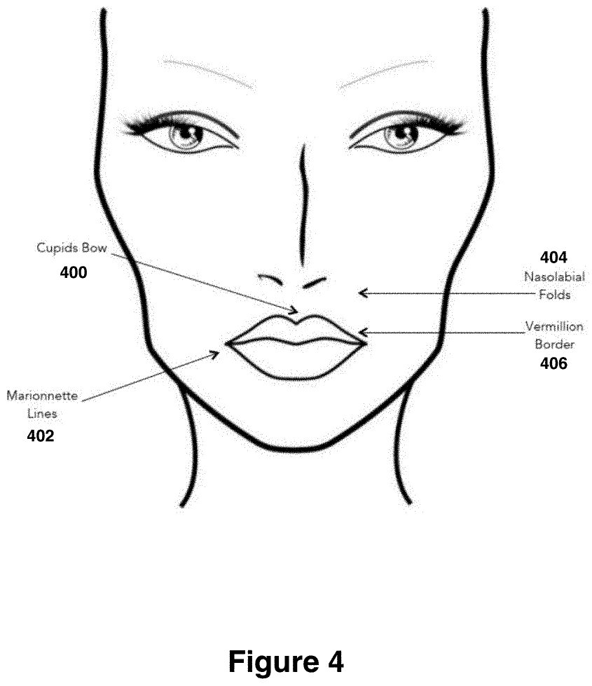

FIG. 4 is a diagram of the primary facial application areas of the present invention. In accordance with the preferred embodiment of the present invention, the primary facial application areas include: the cupid's bow 400; marionette lines 402; nasolabial folds 404; and the vermillion border 406. The present invention can be used to treat dermatological concerns such as: firming and filling wrinkles, nasolabial folds 404, and marionette lines 402; and lip enhancement. This treatment typically lasts approximately from six to nine months and sometimes longer, depending on the Hyaluronic acid used in the treatment and how quickly it breaks down in the recipient's skin. The novel techniques used in the application process of the hyaluron pen are demonstrated to trainees and incorporate practice on live models throughout the day. This training technique includes the following treatments: firming and filling wrinkles, nasolabial folds 404, marionette lines 402; and lip enhancement. According to the present invention, in the alternative, a computer may be used to keep track of clients and their skin conditions, and their outcomes according to the present invention.

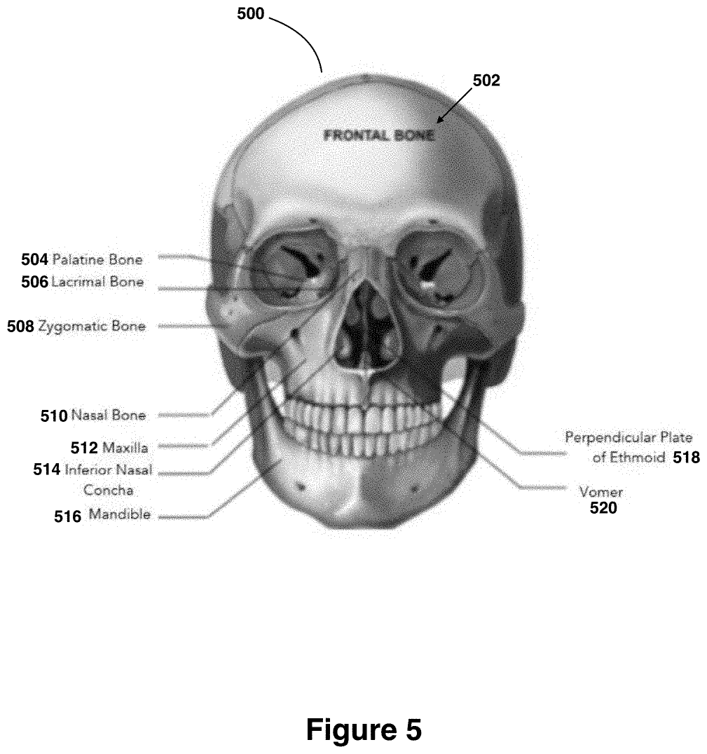

FIG. 5 is a diagram of facial anatomy and physiology pertaining to bone structure as used in the training process of the present invention. In accordance with the preferred embodiment of the present invention, the facial skeleton serves to protect the brain; house and protect the sense organs of smell, sight, and taste; and to provide a frame on which the soft tissues of the face can act to facilitate eating, facial expression, breathing, and speech. The image shown in FIG. 5 provides an overview of the anterior features of the skull 500. These features include the frontal bone 502; palatine bone 504; lacrimal bone 506; zygomatic bone 508; nasal bone 510; maxilla 512; inferior nasal concha 514; mandible 516; perpendicular plate of ethmoid 518; and the vomer 520. The primary bones of the face are the mandible 516, maxilla 512, frontal bone 502, nasal bones 510, and zygomatic bone 508.

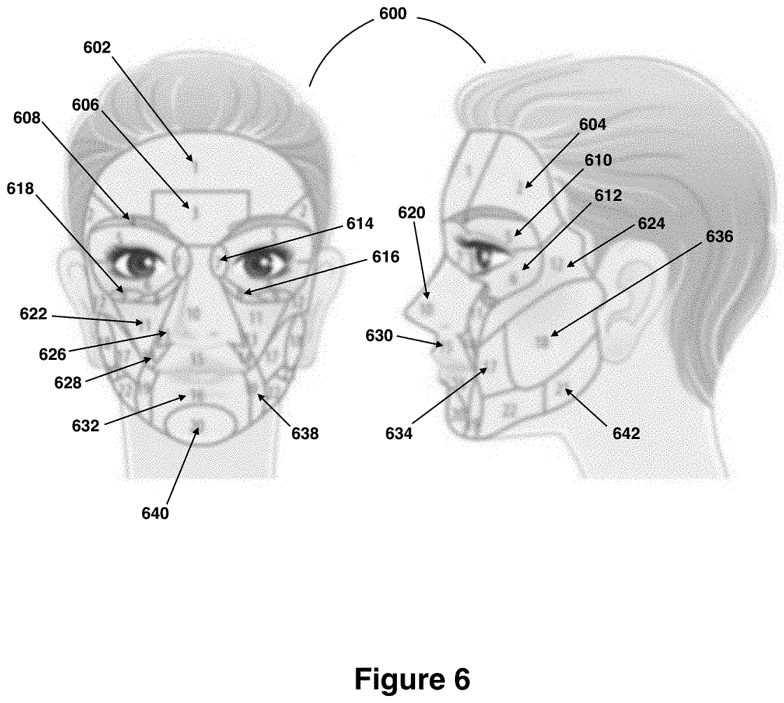

FIG. 6 is a diagram of facial anatomy and physiology pertaining to the regions of the face as used in the training process of the present invention. In accordance with the preferred embodiment of the present invention, having a thorough understanding of the anatomy of the face 600 is important when using Hyaluronic acid. Knowledge of the main arteries in the face 600 can reduce the risk of possible complications with filling techniques. As shown in FIG. 6, the face 600 is divided into 21 regions, with a view to adjusting the areas where dermal filler is performed: frontal 602; temporal 604; glabellar 606; eyebrow 608; upper eyelid 610; lower eyelid 612; nasociliary 614; nasojugal sulcus 616; eyelid lateral sulcus 618; nasal 620; malar 622; zygomatic 624; canine fossa 626; nasolabial sulcus 628; upper lip 630; lower lip 632; cheek 634; preauricular 636; labiomental sulcus 638; mentonian 640; and posterior mandibular region 642.

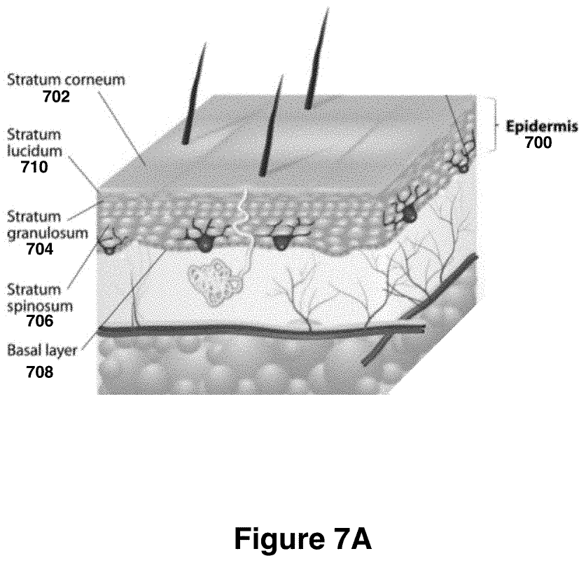

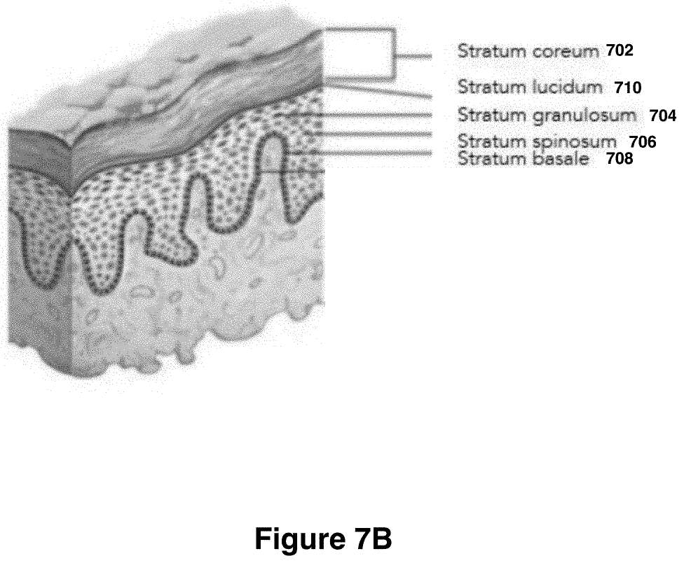

FIGS. 7A-B are diagrams of the internal and external structure of skin as used in the training process of the present invention. In accordance with the preferred embodiment of the present invention, the skin is the largest organ in the body and consists of three layers: the epidermis; the dermis; and the hypodermic or subcutaneous fatty tissue. The skin has three main functions: protection, regulation and sensation. The skin has very important vital functions for keeping the physiological and biochemical conditions of the body in its optimum state. The most important functions of the skin are: regulation of body temperature; preventing the loss of essential body fluids and penetration of toxic substances; protecting the body from harmful effects of the sun and radiation; and excretion of toxic substances with sweat. As shown in FIGS. 7A and 7B, the epidermis 700 is the outer most layer of skin that acts as a barrier preventing toxic chemical and other materials from penetrating deeper into the skin. This is relatively porous and undergoes changes in thickness in response to different factors such as trauma or pressure. The layers of the epidermis 700 differentiate and gradually develop to a more rigid structure, which provides a barrier to excessive loss of body fluids and the penetration of noxious substances. The epidermis 700 has five main layers: Stratum Corneum 702; Stratum Granulosum 704; Stratum Spinosum 706; Stratum Basale (basal layer) 708; and Stratum Lucidum 710. The Stratum Corneum 702 is composed of dead cells which are shed so often that the human body has a completely new outer layer approximately every 35 days. The Stratum Granulosum 704 is the waterproofing layer of the skin. It helps prevent fluid loss and also assists with regulating body temperature. Below the Stratum Granulosum 704 is the Stratum Spinosum 706, which consists of microcells that are filled with Keratin. Keratin cells are also present in the Stratum Basale 708, the deepest layer of the epidermis. The keratin and basal cells divide continually, creating new cells and pushing old dead cells to the surface. This process promotes skin regrowth.

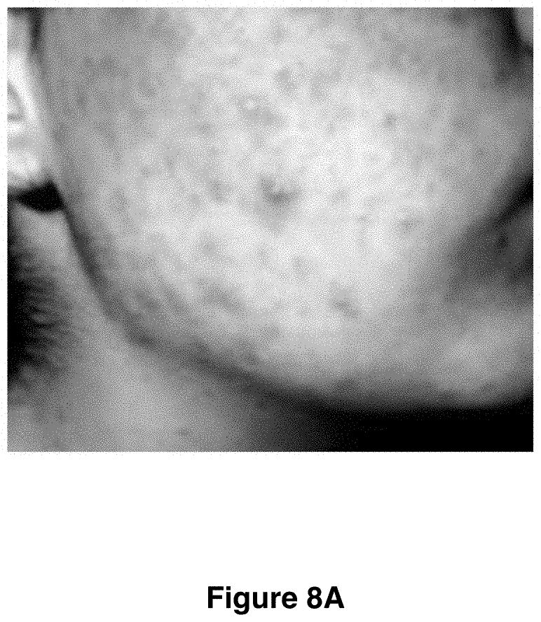

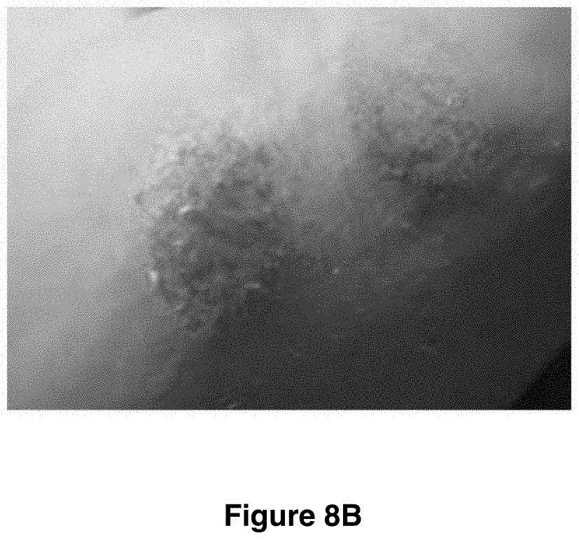

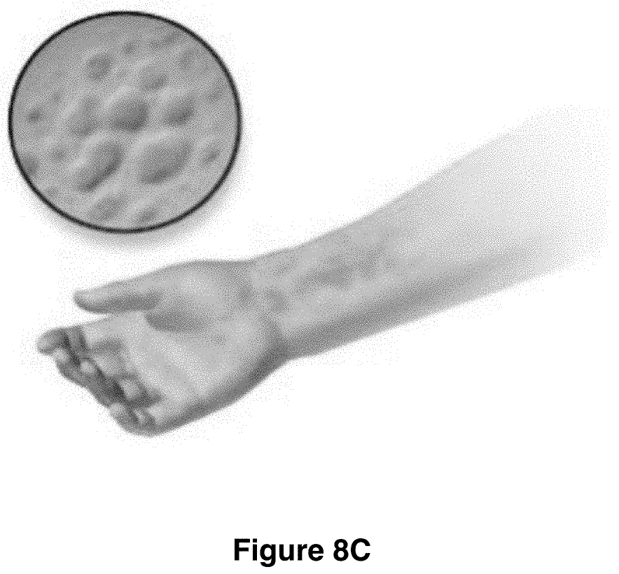

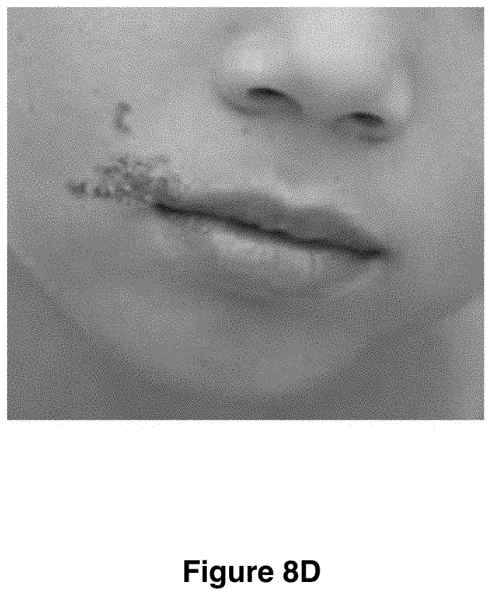

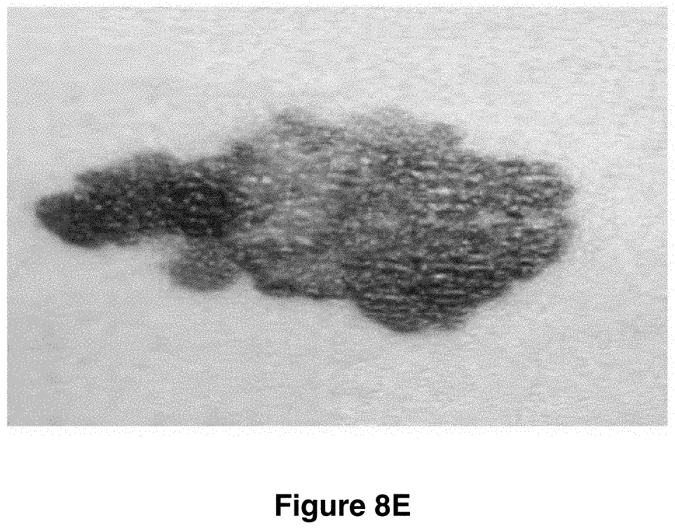

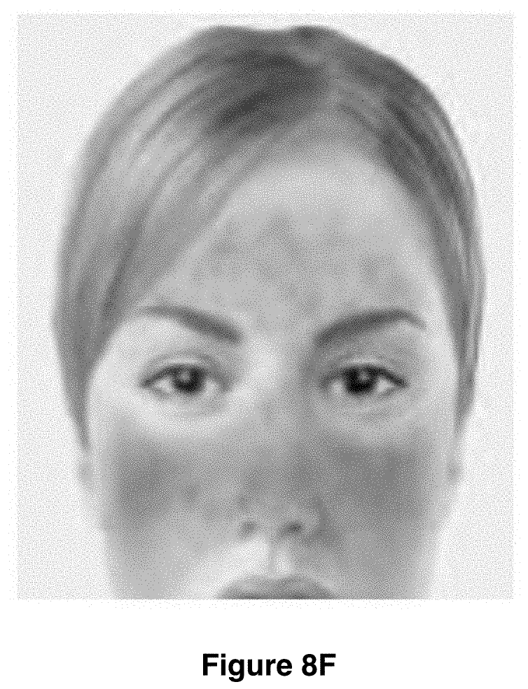

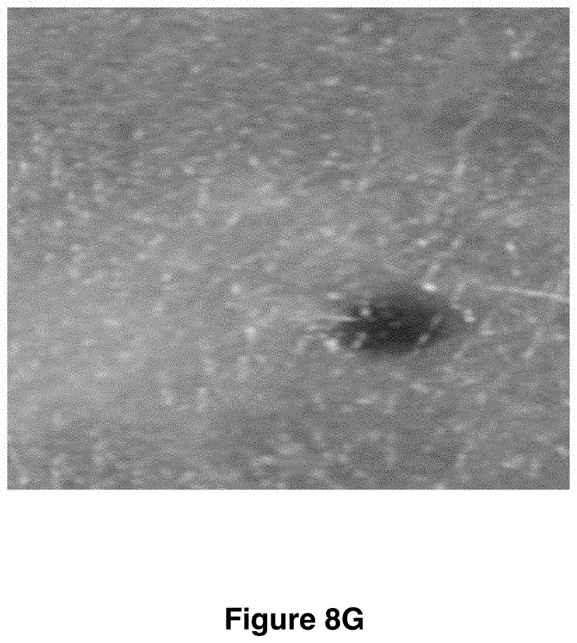

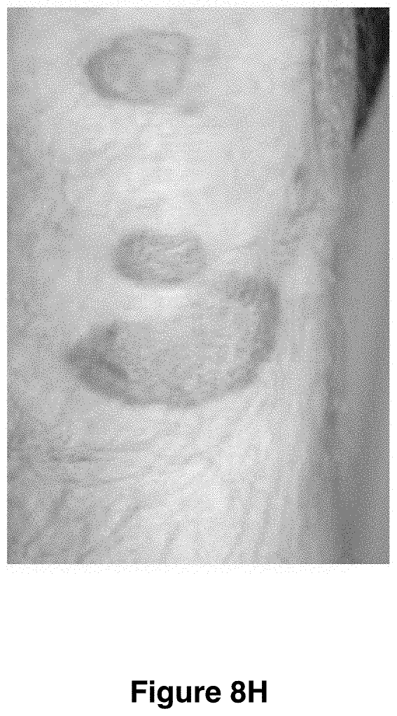

FIGS. 8A-I are images of common skin disorders as used in the training process of the present invention. In accordance with the preferred embodiment of the present invention, it is vital to be able to easily recognize and differentiate between the common skin disorders shown in FIGS. 8A-I in order to avoid potential hyaluron pen treatment complications.

FIG. 8A is an image of acne, which is a condition that affects the skin's oil glands. The small holes in your skin (pores) connect to oil glands under the skin that make a substance called sebum. The pores connect to the glands by a canal called a follicle, and when the follicle of a skin gland clogs up, a pimple grows. Acne is the most common skin condition, with estimated 80 percent of all people experiencing acne at some point. Early treatment or acne is the best way to prevent scars.

FIG. 8B is an image of eczema, also known as atopic dermatitis, which is a long-term skin condition. The most common symptoms are dry and itchy skin, rashes on the face, inside the elbows, behind the knees, and on the hands and feet. There is no single test to diagnose eczema, with medical professionals relying on family medical history information to reach a diagnosis.

FIG. 8C is an image of hives, which are red and sometimes itchy bumps on your skin, usually caused by an allergic reaction to a drug or food, but can also be caused by infections or stress. People who have other allergies are more likely to get hives. Hives usually subside in time but, in serious cases, medical assistance should be sought.

FIG. 8D is an image of impetigo, which is a skin infection caused by bacteria. Usually the cause is staphylococcal (staph), but can also be caused by streptococcus (strep). It is most common in children between the ages of 2 and 6. Impetigo usually starts when bacteria invade a break in the skin (such as a cut, scratch or insect bite). Symptoms start with red or pimple-like sores surrounded by red skin. These sores usually occur on the face, arms, and legs. The sores fill with pus, then break open after a few days and form a thick crust. Impetigo is treatable with antibiotics.

FIG. 8E is an image of melanoma, which is a severe and potentially life-threatening skin cancer. The “ABCD's” of what to watch for with moles on the skin include: asymmetry (the shape of one half does not match the other); border (edges are ragged, blurred, or irregular); color (uneven and may include shades of black, brown and tan; and diameter (change in size, usually an increase). People with melanoma may have surgery, chemotherapy, biological therapy, radiation therapy, or a combination of those treatments.

FIG. 8F is an image of rosacea which is a frequent redness (flushing) of the face, small red lines under the skin, inflamed eyes and/or eyelids, swollen nose and thicker skin. A physician can usually diagnose rosacea with a thorough medical history and physical diagnosis.

FIG. 8G is an image of moles, which are growths on the skin. They happen when cells in the skin, called melanocytes, grow in a cluster with tissue surrounding them. Most people have between 10 and 40 moles and a person may develop new moles from time to time, usually until about the age of 40. About 10% of people have at least one unusual (or atypical) mole that looks different from an ordinary mole. These may be more likely than ordinary moles to develop into melanoma, a type of skin cancer. A healthcare professional should check your moles if they look unusual, grow larger, change in color or outline, or in any other way.

FIG. 8H is an image of psoriasis, which is a skin condition that causes scaling and swelling. Most psoriasis causes patches of thick, red skin with silvery scales. that can itch or feel sore. These patches are often found on the elbows, knees, other parts of the legs, scalp, lower back, face, palms, and soles of the feet but can also appear on other areas. Psoriasis can be hard to diagnose because it can look like other skin conditions. A doctor might need to look at a small skin sample under a microscope in order to confirm the condition. Treatment depends on how serious the disease is, the size of the psoriasis patches, the type of psoriasis and how the patient reacts to certain treatments.

FIG. 8I is an image of a rash, also known as basic dermatitis, which is dry and itchy skin, rashes on the face, inside the elbows, behind the knees, and on the hands and feet. Medical professionals can advise on a good skin care routine, as well as providing advice on how to avoid substances that lead to flares and how to treat symptoms when they occur.

The novel training process of the present invention involves the incorporation of practical considerations for technicians, beginning with an overview of what equipment is needed for the procedure of the present invention. The necessary equipment includes: a beauty bed or chair at correct height; a bed roll, trolley and waste bin, sharps; correct attire, clinical, hair tied back and no jewelry; recipient consent forms; recipient aftercare forms; hyaluron pen starter kit that includes the device and container: at least 5 cartridges/5 hyaluronic acid (1 ml); the training manual; disposable aprons; disposable face masks; and disposable hair nets. The environment required for the procedure of the present invention must include the following: clear, bright lighting; proper sanitation and a sink to wash hands; and a camera for before and after pictures. The procedure of the present invention requires implementing rigorous safety standards, and

all disposables should be disposed of in the correct appropriate containers, with any waste to be collected by a clinical waste company. The treatment room for the procedure of the present invention your is to be thoroughly cleaned with CaviCide wipes and virucidal disinfectant. Between each recipient, all organisms and blood borne pathogens should be removed.

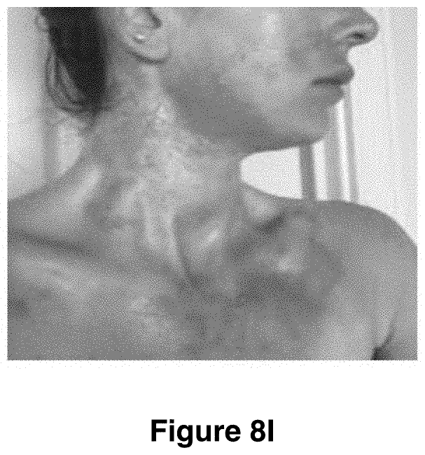

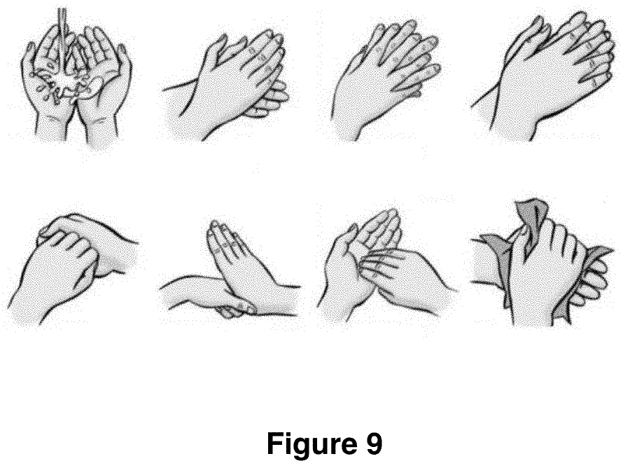

FIG. 9 is a hand washing diagram as used in the training process of the present invention. In accordance with the preferred embodiment of the present invention, hand washing, and drying with disposable paper towels, between each recipient is essential for the procedure of the present invention. The proper hand washing technique as shown in FIG. 9 is part of the training procedure of the present invention. This is to insure that there is no dangerous cross-contamination, which is the process by which bacteria or other micro-organisms are unintentionally transferred from one substance or object to another, with harmful effect. Hand washing is the most important step in preventing cross-contamination.

When performing the hyaluron pen procedure of the present invention, a technician must always wash their hands thoroughly before and after: eating and drinking; smoking; applying cosmetics; preparing food; working with a previous recipient; and using a mobile phone. Technicians must also wash their hands after handling waste and contaminated equipment. When performing the hyaluron pen procedure, the recipient of the treatment should be sat comfortably on a beauty bed or treatment couch. To avoid bacteria from spreading from recipient to recipient, technicians must dispose of the used bed roll and replace it after each use. The following items all hold a risk of cross-contamination: disposable masks; hair nets; gloves; aprons; towels; gauze pads; and cartridges. Once used, these items should be disposed of in a clinical waste bin and the technician's hands must be washed.

Another component of the training process of the present invention is recipient consultations and factors affecting treatment longevity. The treatment of the present invention typically lasts approximately from six to nine months and sometimes longer, depending on the Hyaluronic acid used in the treatment and how quickly it breaks down in your recipient's skin. The technician must be clear when advising recipients on the possible outcomes of the procedure and to be able to identify recipients that are not suitable for hyaluron pen treatment. Treatment longevity can depend on factors such as: age; lifestyle; and size, symmetry and shape of the recipient's natural features. If your recipient has had lip injections before and the filler has not lasted, it is likely that the Hyaluronic acid used via the hyaluron pen treatment will not last either. This treatment will not be effective if the recipient has no visible lines, or very deep lines present. The treatment recipient's skin, and the subsequent effectiveness of a hyaluron pen treatment, can be affected by a range of lifestyle factors. The most relevant issues for a technician to consider when evaluating and advising a recipient are: sun exposure; lack of sleep; stress and unhealthy lifestyle; air pollution; smoking; alcohol consumption; and nutritional deficiencies. A Consultation Form must be filled out by every recipient prior to a treatment. The completed Consultation Form will highlight any allergies or medical conditions the recipient has that will prevent the recipient from undergoing a hyaluron pen treatment. It also gives the recipient the opportunity to talk through the treatment and ask the hyaluron pen technician any questions.

Another component of the training process of the present invention is to teach the technician to identify and determine procedure contraindications on the use of hyaluron pen. A potential recipient with any of the following conditions must not receive a hyaluron pen treatment: recipient is under the age of 18; diabetes; pregnancy or breastfeeding; recipient has glaucoma or is taking blood thinning medicines (e.g., aspirin, warfarin); presence of skin diseases such as psoriasis or eczema; inflammations around the area to be treated; transmittable blood conditions such as HIV or Hepatitis; haemophilia; healing disorders or uncontrolled high blood pressure; recipient is taking any skin medication such as Accutane or steroids; active skin cancer; recipient is undergoing radiotherapy or chemotherapy; epilepsy; recipient has a pacemaker or major heart problems; recipient has had Botox within one month of the proposed treatment (only when used in same area); presence of cold sores or herpes; and recipient has an allergy to Lidocaine (Can receive treatment, but lidocaine numbing cream must be avoided).

In addition, the recipient cannot have previously undergone HA filler treatment in the proposed area within the past three months. The complications of this can be moving of the filler when trying to create a perfect shape, as well as making the injection site bigger due to the stretched skin, resulting in the recipient suffering from bleeding and bruising. It is essential to obtain a full medical treatment history from every recipient before proceeding. The recipient will be able to state any medical conditions on the consultation form on the day of the proposed hyaluron pen treatment. Under no circumstances should any hyaluron pen treatment be carried out if the recipient or technician has any of the above concerns.

Immediately after the treatment, the recipient may experience tenderness, swelling and slight bruising. This is normal and will subside within a few hours to a few days after the treatment. Redness to the treated area is also normal and will only last a few hours. Within the first 24 hours of a hyaluron pen treatment, the recipient may develop lumps. This is the Hyaluronic acid absorbing water from the body and can be easily gently massaged away between a finger and thumb 2 weeks later. Swelling and bruising are the most common side-effects following treatment. These symptoms will subside within a week, depending on the individual. If a recipient develops a haematoma (large bruising), this could last up to ten days. Applying Arnica ointment to the treated area can help aid bruising and speed up the healing process. Avoid applying make-up over the treated area for up to twelve hours. This is reduce the risk of infection. Exposure to UV light, sunlight and saunas are not recommended for 48 hours following treatment. If the recipient suffers from facial cold sores, there is a possibility that the treatment they have received could create an outbreak. Other risks following hyaluron pen treatment, although extremely rare, include but are not limited to: discoloration of the treated area; necrosis (death of skin); abscess formation; granulomas (abnormal growth of skin); and infection.

The following complications are risks seen with hypodermic needle treatments and are extremely rare when working with hyaluron pen. Skin necrosis, which is the death of cells in living tissue caused by external factors such as infection, trauma, or toxins. Technicians should note that necrosis occurs when compression is used or HA filler is inserted into the blood vessel itself. This can sometimes be a concern for a technician when injecting HA via hypodermic needle into an area, but occurrences of skin necrosis resulting from hyaluron pen treatment will be extremely rare because it is practically impossible to cause this type of trauma with a needle-free device. Hypersensitivity, which can happen and this will result in swelling and redness in the area. The Tyndall effect, which occurs when HA fillers are inappropriately implanted into the superficial dermis or epidermis. When light hits the surface of the skin with superficially-placed HA particles, the particles cause the light waves to be reflected and due to increased scatter, blue is the predominant color that emerges. Although rare, this can happen if the Hyaluron pen is not pressed at 90 degrees to the skin and too much Hyaluronic acid is inserted to the upper part of the dermal. Vascular occlusion can occur when filler is injected into an artery or when a sufficient quantity is injected near an artery so as to cause a compression blockage. However, technicians must be aware when evaluating and advising a recipient that the elderly are, in general, more likely to develop this sort of complication. As a needle-free device, occurrences of vascular occlusion will be extremely rare when working with hyaluron pen.

Without the invasive element of a hypodermic needle, infections resulting from hyaluron pen treatments will be extremely rare, although possible. A recipient might experience complications resulting from chronic bacteria activation, such as oral herpes. In order to avoid such infections, recipients with a history of oral herpes should treat the situation regardless of active complications for two weeks prior to hyaluron pen treatment. Anaphylaxis is the most severe form of allergic reaction a recipient can have. Recipients with an allergic reaction often have visible skin symptoms such as swelling of the face, the lips or the eyes. Once it involves their breathing and/or their heart and the situation becomes dangerous, and life threatening, then this would be classed as anaphylaxis. A recipient who is experiencing an anaphylactic episode will appear pale, wheezy, dizzy and short of breath. The technician must act quickly if the signs of anaphylaxis are present. The technician should carry out the following steps when dealing with a recipient suffering from anaphylaxis: ensure that the recipient remains as still as possible; and the recipient should be lying down and if they are feeling weak, dizzy or appear pale and sweating their legs should be raised. When dialing 911, technicians should inform emergency services that the recipient is suffering from anaphylaxis, and give clear and precise directions to the emergency operator, including the address of the location. If adrenaline has been given, the technician must make a note of the time this was administered. A second dose can be given after five minutes, if there has been no improvement. If the recipient's condition deteriorates after making the initial 911 call, a second call to the emergency services should be made to ensure an ambulance has been dispatched. The technician must someone outside to direct the ambulance crew upon arrival and try to ascertain the substance that caused the reaction and ensure that the ambulance crew has been informed.

Part of the initial assessment and past medical history of the recipient is to ascertain whether they have any sensitivity to Lidocaine, an anaesthetizing ingredient in a number of popular HA fillers. Filler containing Lidocaine is not needed for treatment and as a general rule, any technician without prior aesthetic training would be advised to use products that do not contain Lidocaine.

Another component of the training process of the present invention is the process of removing hyaluronic acid filler with Hylaronidase. There may be treatments when the recipient dislikes the overall look of the results or when filler has been injected in the wrong place, due to the natural asymmetry of the human face. It is important when working hyaluron pen that a technician also understands how filler can be removed, if necessary. In the U.S., Hyaluronidase is a license prescription-only medicine. It must only be administered by a medical professional and with the recipient's fully informed consent and where the medical professional believes that it would be in the best interests of the recipient to do so. Research suggests that the enzyme Hyaluronidase has no effect on the patient's own hyaluronic acid and risks appear to be few, however a very small percentage of recipients may be allergic to the enzyme. Hyaluronidase comes in 1 ml vials and is mixed with sterile saline for injection. It is used in tiny quantities, depending on how much HA filler needs to be dissolved. When considering the quantity of Hyaluronidase for administration, the technician must inform the medical professional of the type of HA filler used in the original treatment, the quantity that was used and the area that has been treated. Technicians must be aware that, at the present time, there is no scientific data indicating the exact quantity of Hyaluronidase to use; it is a matter of judgment and experience. It is suggested that the enzyme Hyaluronidase quickly breaks down the extracellular Hyaluronic acid filler, thus causing it to dissolve, although there are currently no proven studies to demonstrate the effectiveness of Hyaluronidase in dissolving Hyaluronic Acid gels.

Another component of the training process of the present invention is the set up of business and advertising for the technician. Pricing must be considered when starting a hyaluron pen procedure business, starting with research into the prices charged by local salons, and how long they have been providing treatments. Every hyaluron pen treatment will use 1 ampoule cartridge per procedure and should be used fully and disposed of following use. Pricing of the procedure must also be established by the technician, taking the cost of each cartridge into account. The advertising and marketing component of the business operation includes taking as many before-and-after pictures as possible, in order to build a wide portfolio of recipients. A water mark of the technician's logo is recommended to be used on all of images that are uploaded to social media for promotional purposes. Uploading success stories and before-and-after images or video montages can have a powerful effect in the world of social media. It is recommended that the technician has clear, bright lighting for all of the images and videos. For marketing purposes, it is important to appreciate that recipients of the procedure will provide personal data. This data must be kept secure, used only for the purposes it was provided, and it must be deleted when that use has ended. A photo release form signed by the recipient is recommended as part of the novel training procedure.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A system using a hand-held pen device and observing client skin conditions comprising:
a hyaluronic pen containing hyaluronic acid;
a clinical technician using said hyaluronic acid filled pen to eject hyaluronic acid from said hyaluronic pen so that said hyaluronic acid is inserted under the outermost layer of skin of a client undergoing a skin treatment, wherein said clinical technician applies more pressure to said hyaluronic pen against said outermost layer of skin to enable a greater amount of hyaluronic acid to be inserted under said outermost layer of skin; and
wherein said clinical technician may vary the speed at which said hyaluronic pen traverses said outermost layer of skin so that amounts of said hyaluronic acid inserted under said outermost layer of skin may be varied in accordance with skin conditions observed by said clinical technician.

2. The system of claim 1, wherein said hyaluronic acid filled pen device is pressed firmly against said outermost layer of skin until said clinical technician achieves a desired amount of hyaluronic acid under said outermost layer.

3. The system of claim 1, wherein said clinical technician insures that a vacuum seal is formed between said pen and said outermost layer of the skin to minimize the amount of air introduced beneath said outermost layer of skin.

4. The system of claim 1, wherein a dermatological concern includes firming skin.

5. The system of claim 1, wherein a dermatological concern includes filling wrinkles.

6. The system of claim 1, wherein a dermatological concern includes forming nasolabial folds.

7. The system of claim 1, wherein a dermatological concern includes forming marionette lines.

8. The system of claim 1, wherein a dermatological concern includes firming skin.

9. The system of claim 1, wherein a dermatological concern is addressed by said clinical technician in accordance with observed skin conditions.

10. The system of claim 9, wherein deeper wrinkles are addressed with corresponding more force applied to said pen by said technician.

11. The system of claim 9, wherein deeper wrinkles are addressed with corresponding slower speed of application applied to said pen by said technician.

12. A method of using a hand-held pen device and observing client skin conditions comprising:
a clinical technician using a hyaluronic acid filled pen containing hyaluronic acid;
said clinical technician using said hyaluronic pen to eject hyaluronic acid from said hyaluronic pen so that said hyaluronic acid is inserted under the outermost layer of skin of a client undergoing a skin treatment, wherein said clinical technician applies more pressure to said hyaluronic pen against said outermost layer of skin to enable a greater amount of hyaluronic acid to be inserted under said outermost layer of skin; and
wherein said clinical technician may vary the speed at which said hyaluronic pen traverses said outermost layer of skin so that amounts of said hyaluronic acid inserted under said outermost layer of skin may be varied in accordance with skin conditions observed by said clinical technician.

13. The method of claim 12, wherein said hyaluronic acid filled pen device is pressed firmly against said outermost layer of skin until said clinical technician achieves a desired amount of hyaluronic acid under said outermost layer.

14. The method of claim 12, wherein said clinical technician insures that a vacuum seal is formed between said pen and said outermost layer of the skin to minimize the amount of air introduced beneath said outermost layer of skin.

15. The method of claim 12, wherein a dermatological concern includes firming skin.

16. The method of claim 12, wherein a dermatological concern includes filling wrinkles.

17. The method of claim 12, wherein a dermatological concern includes forming nasolabial folds.

18. The method of claim 12, wherein a dermatological concern includes forming marionette lines.

19. The method of claim 16, wherein more force is applied upon said pen by said clinician corresponding with wrinkle depth.

20. A system using a hand-held pen device and observing client skin conditions comprising:
a hyaluronic pen containing hyaluronic acid;
a clinical technician using said hyaluronic acid filled pen to eject hyaluronic acid from said hyaluronic pen so that said hyaluronic acid is inserted under the outermost layer of skin of a client undergoing a skin treatment, wherein said clinical technician applies more pressure to said hyaluronic pen against said outermost layer of skin to enable a greater amount of hyaluronic acid to be inserted under said outermost layer of skin;
wherein said clinical technician may vary the speed at which said hyaluronic pen traverses said outermost layer of skin so that amounts of said hyaluronic acid inserted under said outermost layer of skin may be varied in accordance with skin conditions observed by said clinical technician; and
wherein said clinical technician utilizes a computer and associated database to assist in determining a manner of use of said hyaluronic acid filled pen device upon said outermost layer of skin.
